(19) United States
(12) Patent Application Publication
(10) Pub. No.: US 2022/0062552 A1
(43) Pub. Date: Mar. 3, 2022

(54) REMOTE MANAGEMENT OF A MEDICAMENT DELIVERY DEVICE USING DATA ANALYTICS

(71) Applicant: Insulet Corporation, Acton, MA (US)

(72) Inventors: Ashutosh Zade, San Diego, CA (US); Jay Jantz, Acton, MA (US); Yibin Zheng, Hartland, WI (US); Trang Ly, Concord, MA (US); Joon Bok Lee, Acton, MA (US); Jason O'connor, Acton, MA (US); Ian Mclaughlin, Groton, MA (US); Nicholas Conte, Harvard, MA (US); Steven Cardinali, Tewksbury, MA (US)

(21) Appl. No.: 17/446,084

(22) Filed: Aug. 26, 2021

Related U.S. Application Data
(60) Provisional application No. 63/070,905, filed on Aug. 27, 2020.

(51) Int. Cl. A61M 5/172; G16H 20/17

(52) U.S. Cl. CPC A61M 5/1723; A61M 2005/14208; G16H 20/17

(57) ABSTRACT

Exemplary embodiments may perform data analytics on data regarding a user of a medicament delivery device and data regarding other users of like medicament delivery devices to provide insights and guide management of the medicament delivery device for the user. The data analytics may be so called “big data” analytics that are performed on large data sets. The data analytics may be performed on various types of data to help determine what actions, if any, the remote management should take. The data analytics may be performed by the software on the remote management device or by other computational resources, such as by other servers or cloud computing resources. Exemplary embodiments may provide for remote management of a medicament delivery device. The management may be remote in that the management is performed via a remote management device, such as a computing device, that is remotely located from the medicament delivery device.

### CROSS-REFERENCE TO RELATED APPLICATIONS

This application claims the benefit to U.S. Provisional Application No. 63/070,905, filed Mar. 24, 2021, the entire contents of which are incorporated herein by reference in their entirety.

### BACKGROUND

Many medicament delivery devices are meant to be worn by users so as to be able to deliver medicaments over time to the users while allowing the users to go about their daily activities. In many instances, the delivery of the medicaments by the medicament delivery devices is done automatically under programmatic control. Specifically, for each such medicament delivery device, a programmatic control method may be provided to determine when a medicament should be delivered, when a medicament should not be delivered and how much medicament is delivered. Typically, the programmatic control method has parameter values that drive the decisions regarding when to deliver a medicament and how much medicament to deliver. In some conventional medicament delivery systems, the initial settings for the parameter values are set to a population normal set of values. In other words, a “one size fits all” approach is used. This has the drawback that the normal set of values may not work well for all users of a medicament delivery device. Some control methods are adaptive and adjust the parameter values over time to more optimal values. Unfortunately, this may often take a good bit of time. As a result, some users must endure sub-optimal parameter values for an undesirably long period of time.

### SUMMARY

In accordance with an exemplary embodiment, a method is performed with a processor. Per the method, other users of a medicament delivery device having adaptivity characteristics like a selected user of the medicament delivery device are identified. With the processor, an adaptivity rate for one or more parameters of the medicament delivery device for the selected user is selected based on data related to adaptivity rates for the identified other users. The one or more parameters play a role in regulating automatic medicament delivery by the medicament delivery device. The processor takes an action to set the adaptivity rate for the medicament delivery device to the selected adaptivity rate.

Instructions for performing the method may be stored on a non-transitory storage medium.

The action the processor takes in the above-described method may include sending a communication to the medicament delivery device of the selected user or sending a command to set the adaptivity rate as the selected adaptivity rate for the medicament delivery device of the selected user. The selected adaptivity rate may be for a single parameter. The medicament delivered by the medicament delivery device may be insulin, glucagon-like peptide-1 (GLP-1) agonists, pramlintide or other glucose affecting or regulating medicaments. The adaptivity characteristics may be one or more of the following metrics where the medicament is insulin: discrepancy between amount of insulin delivered automatically by the medicament delivery device for a day versus total daily insulin, a ratio of amount of insulin requested as boluses for a period versus an amount of automatically delivered insulin for the period, or a ratio of a percentage of time where glucose is below a hypoglycemic threshold versus a percentage of time where automatic delivery of insulin was suspended.

The method may include categorizing the data related to adaptivity rate for the identified users into categories. The method may also include choosing a matching category among the categories for the selected user and using the data related to adaptivity rate for the identified users in the matching category in the selecting of the adaptivity rate for one or more parameters of the medicament delivery device for the selected user. The method may include projecting an expected final value for the one or more parameters and determining an adaptivity rate based on how great of a difference there is between the expected final value and an initial value of the one or more parameters. The determined adaptivity rate may be the selected adaptivity rate. The categorizing may categorize the data by glucose control outcomes, and the choosing of a matching category may entail choosing the matching category as one that has a glucose control outcome that suits the selected user. The categorizing may categorize based on data including at least one of mean glucose value or a percentage of time where the glucose value for the selected user is in an acceptable range. The selecting of an adaptivity rate for one or more parameters of the medicament delivery device for the selected user may also be based on at least one of date, time, day of week or whether a day is a holiday or part of a holiday season. The method may include updating the data related to adaptivity rate for the identified user before the selecting of the adaptivity rate.

In accordance with an exemplary embodiment, a method is performed in which information regarding a user of a medicament delivery device and/or the medicament delivery device is received at a remote computing device. The remote computing device may not be connected to a network to which the medicament delivery device is connected. A communication is sent to the medicament delivery device from the remote computing device causing at least one of the following: a medicament to be delivered by the medicament delivery device to the user, settings of the medicament delivery device to be changed, settings of a controller of the medicament delivery device to be changed or information to be sent to the user of the medicament delivery device.

The communication may be sent to the controller for the medicament delivery device.

The medicament delivery device may be an insulin delivery device that delivers insulin to the user.

A user interface may be displayed on the remote computing device for initiating the sending of the communication. The received information may include glucose information for the user. The method may also include displaying at least some of the received information on a display of the remote computing device. A questionnaire may be sent or a user interface may be provided to the user to solicit the information that is received. The solicited information may include at least one of information regarding activities of the user, information regarding hobbies of the user, information regarding eating habits of the user, information about the health of the user, information on medicament delivery by the user, information regarding the blood glucose history of the user or demographic information about the user. The method may further include analyzing the received information and programmatically determining to send the communication based on the analyzing. The analyzing may include performing data analytics on data for other users of the medicament delivery device.

Instructions for performing the method may be stored on a non-transitory storage medium.

### BRIEF DESCRIPTION OF THE DRAWINGS

FIG. 1 depicts a block diagram of an illustrative medicament delivery system for an exemplary embodiment.

FIG. 2 depicts a more detailed block diagram of the local manager 104 of FIG. 1.

FIG. 3 depicts a more detailed block diagram of the medicament delivery device 102 of FIG. 1.

FIG. 4 depicts a flowchart of illustrative steps that may be performed to set an adaptivity rate in an exemplary embodiment.

FIG. 5A depicts a flowchart of steps that may be performed to generate categories based on adaptivity characteristics.

FIG. 5B depicts a flowchart of illustrative steps that may be performed to generate categories based on glucose control outcomes.

FIG. 6 depicts an illustrative table of adaptivity characteristics and other data for users.

FIG. 7 depicts a flowchart of illustrative steps that may be performed to choose an adaptivity rate where a category has multiple associated adaptivity rates.

FIG. 8 depicts a block diagram of illustrative types of actions that may be taken as part of step 408 of FIG. 4 in an exemplary embodiment.

FIG. 9 depicts a flowchart of illustrative steps that may be taken in an exemplary embodiment in response to receiving data regarding a user.

FIG. 10A is a flowchart depicting illustrative steps where a questionnaire is used to solicit data.

FIG. 10B is a flowchart depicting illustrative steps where a user interface is used to solicit data.

FIG. 10C is a flowchart depicting illustrative steps that may be performed when data is received via electronic communication.

FIG. 11 depicts a block diagram of types of data that may be solicited regarding a user.

FIG. 12 depicts a block diagram of types of communication that may be sent based on analytics of received data regarding a user.

FIG. 13 depicts a block diagram of illustrative types of parties that may originate communications to the local manager in an exemplary embodiment.

### DETAILED DESCRIPTION

Exemplary embodiments may perform data analytics on data regarding a user of a medicament delivery device and data regarding other users of like medicament delivery devices to provide insights and guide management of the medicament delivery device for the user. The data analytics may be so called “big data” analytics that are performed on large data sets. The data analytics may be performed on various types of data to help determine what actions, if any, the remote management should take. The data analytics may be performed by the software on the remote management device or by other computational resources, such as by other servers or cloud computing resources. The data analytics may entail, for example, pattern matching, categorization, comparison to thresholds, data mining, artificial intelligence techniques, machine learning, etc.

One example of applying data analytics is that data regarding adaptivity characteristics of a user may be compared to data on the adaptivity characteristics of other users to help decide the values of the parameters of the medicament delivery device that control a rate of adaptivity of a control method for the medicament delivery device. The control method may determine how much medicament is delivered to the user and when (e.g., rate and time). The control method may have a control loop that adjusts control parameters over time based on how close a user's response to the medicament delivery is to a desired response. The rate of adaptivity refers to how quickly the control method adapts to approach the desired response.

Additionally, the data analytics may determine what the initial settings are for a user, how the settings need to be changed for a user, whether a medicament should or should not be delivered to a user via the medicament delivery device, whether constraints should be loosened or tightened, whether a notification or advisory should be sent to a user or a caregiver (e.g., parent/guardian or HCP), etc. The data analytics may improve the quality of the settings of the medicament delivery device. The settings may be better customized based on analyzing data for users that are similar to the user. Moreover, the data analytics may provide useful information regarding when to deliver boluses of a medicament and a best size for each of the boluses. The data analytics may also provide useful information regarding basal delivery rate of a medicament throughout the day or over time.

Exemplary embodiments may provide for remote management of a medicament delivery device. The management may be remote in that the management is performed via a remote management device, such as a computing device, that is remotely located from the medicament delivery device. The remote management device may not have a direct wired or wireless network connection with the medicament delivery device. Instead, the remote management device may communicate over remote network connections, such as over web-based connections (e.g., over the Internet) or over cellular phone network connections, with a locally located controller for the medicament delivery device. The locally located controller may then pass on the communications to the medicament delivery device or may interpret communications from the remote management device and take appropriate action relative to the medicament delivery device based on those communications.

The remote management device may provide additional computational resources and storage resources relative to conventional management systems that rely on resources of the medicament delivery device and/or a handheld local management device. The medicament delivery device and handheld local management device may have limited computational power and limited storage capability. In contrast, the remote management device may have access to substantial computational power and a large storage capacity.

The remote management may entail taking actions such as establishing settings for the medicament delivery device, adjusting settings for the medicament delivery device, initializing the medicament delivery device, causing a medicament to be delivered to the user from the medicament delivery device, causing medicament delivery to the user to cease, providing messages to the user, providing a form or other user interface to the patient for completion, issuing other types of commands to the medicament delivery device, monitoring user activity, monitoring a user's history of the use of the medicament delivery device, monitoring medical data regarding the user, or other types of management activities.

The remote management may be informed by data regarding the user, data from the medicament delivery device, data from the local management device and data regarding other users of the medicament delivery device. The remote management device may solicit information from a user via a questionnaire, via a user interface or via communications like messages.

Medical personnel may remotely monitor the medicament delivery device and data regarding the user. For example, suppose that the medicament delivery device delivers insulin. In that case, a medical professional may monitor the glucose level of the user and deliver boluses of insulin when needed or halt boluses when there is a heightened risk of hypoglycemia. The medical professional may also alter the delivery of basal insulin based on monitored information via the remote management device. Still further, the medical professional may initiate communications with the user via the remote management device. For example, the medical professional may send emails, text messages or other types of communications to the user via an application running on the local management device. The user may respond in kind with communications.

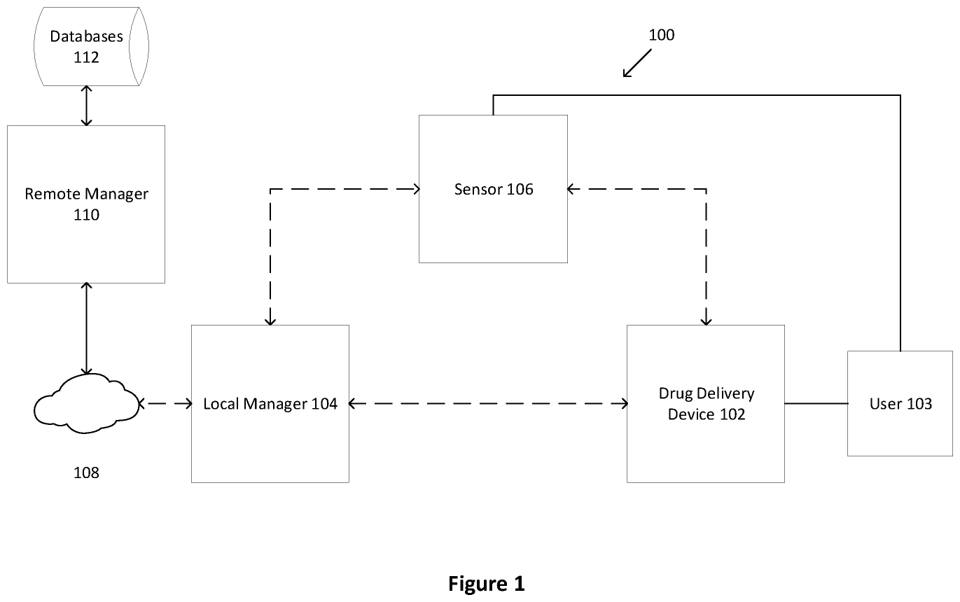

FIG. 1 depicts an illustrative medicament delivery system 100 for an exemplary embodiment. The medicament delivery system 100 includes a medicament delivery device 102 for delivering a medicament to a user 103. The medicament delivery device 102 may, for example, be an insulin pump, patch pump, or other medicament delivery mechanism for delivering insulin to the user 103. In other instances, the medicament delivery device 102 may deliver medicaments, such as painkiller medicaments, therapeutic medicaments or chemotherapy medicaments. The medicament delivery device 102 may be wearable by the user 103 and may be physically connected to the user to facilitate medicament delivery. The medicament delivery system 100 may include a local manager 104 for local management of the medicament delivery device102. The local manager 104 may be a dedicated device that is customized to control the medicament delivery device 102. Alternatively, the local manager 104 may be an application that runs on a smart phone, tablet or other portable computing device. Still further, in some embodiments, the local manager 104 may be integrated with the medicament delivery device 102 in a common device. There may be a wired or wireless connection between the local manager 104 and the medicament delivery device 102 to facilitate bidirectional communications, including commands.

The medicament delivery system 100 also may include a sensor 106 for sensing biological information regarding the user 103. For example, the sensor 106 may sense a glucose level, blood pressure, heart rate, cholesterol level, and other types of biological information that can be sensed by biosensors. The sensor 106 may be wearable by the user, may be implantable or may be a device that is not worn or secured to the user 103. The sensor 106 may have a wired or wireless connection with the medicament delivery device 102. In addition, the sensor 106 also may have a wired or wireless connection with the local manager 104. This connection may be used, for example, to provide sensed biological data for the user 103 to the local manager 104.

The local manager 104 may interface with a network or networks 108, such as a network that provides Internet access, a cellular phone network or a combination of such networks. The local manager 104 may communicate with a remote manager 110 via the network(s) 108. The remote manager 110 provides remote management of the medicament delivery device 102 as will be described in more detail below. The remote manager 110 may be realized via software running on one or more remote computing devices, such as servers, desktop computers or the like. The remote manager 110 may be realized via cloud services running on a cluster on a cloud computing environment. The remote manager 110 may have access to storage, holding large data sets, such as one or more databases 112 of user data for other users of the medicament delivery device 102, stored across one or more storage devices. As was mentioned above, the remote manager 110 may have access to substantially more computational resources and storage resources than the local manager 104. The remote manager 110 may perform big data analytics or prompt the performance of such big data analytics on its behalf.

A wide range of descriptive analytics can be implemented to leverage data that can be gathered in medical devices for improved system performance for users. For example, a simple analytics assessment on incidence of certain clinical events, such as hypoglycemia, can be utilized to derive significant outcomes. A sudden unexplained increase in the rates of hypoglycemia for a certain region of users may indicate that a system component may be defective for the batch of devices that were sent to the user or users, or there is a defective insulin supply with erroneously high concentrations.

Next, a high-level diagnostic analytics assessment can be executed to identify correlation between multiple areas of available data. For instance, a cross-functional assessment on the additional data available for users who experience increased hypoglycemia can be made, including demographics, system use patterns, and location patterns. It may be found that users who are older than 30 years, who bolus often, and/or who travel frequently, are closely associated with an increased rate of hypoglycemia. In this case, the system characteristics on bolusing sequences and time zone change sequences can be reviewed to assess whether there are particular edge cases that may result in increased risk of hypoglycemia.

Further, predictive analytics assessments can also be executed on available data to assess whether there may future events whose severity can be reduced. For example, the characteristics highlighted in the previous paragraph—older than 30 years, bolusing often, and traveling often—can be identified for other users who did not yet experience increased risk of hypoglycemia. Then, education efforts or healthcare provider communications can be executed to reduce the severity of hypoglycemic risk that is experienced by these users.

Finally, prescriptive analytics can be utilized to not only guide the behaviors of the system, but also guide the behaviors of the users. For example, it may be recognized as in the previous example that higher frequency of boluses and increased travel incidence may result in increased risk of hypoglycemia. These analytics can then be fueled to suggest directly to the user, via the system's interface, how to avoid or guide such behaviors, e.g., reduce the frequency of boluses or decrease the frequency of travel.

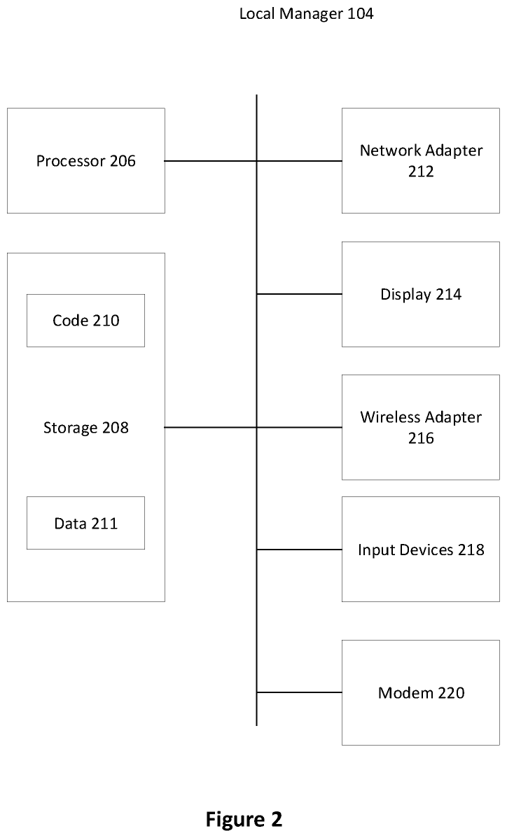

FIG. 2 depicts a block diagram of the local manager 104 showing more detail. The local manager 104 may include a processor 106, such as a Central Processing Unit (CPU), a Graphics Processing Unit (GPU), an Application Specific Integrated Circuit (ASIC), a Field Gate Programmable Array (FPGA) or other type of processor. The local manager 104 may also include a storage 208 for storing code 210 and data 211. The code 210 may be executed by the processor to realize the functionality of the local manager 104 described herein. The data 211 may include biometric data gathered by the sensor 106 as well as other types of data. The storage 208 may include various types of memory including but not limited to Random Access Memory (RAM), Read Only Memory (ROM), Electrically Programmable Read Only Memory (EPROM), Electrically Erasable Programmable Read Only Memory (EEPROM), magnetic storage, optical storage, flash memory, solid state storage, removable storage media and other types of non-transitory computer-readable storage media that store instructions including code 211 that is executable by the processor 206.

The local manager 104 may include a network adapter 212 for interfacing with a network, such as a Local Area Network (LAN). The local manager 104 may include a display 214 for displaying visual output. The display 214 may be, for example, a Light Emitting Diode (LED) display, a Liquid Crystal Display (LCD) or the like. The local manager 104 may include a wireless adapter 216, for interfacing with a wireless network, like a Wi-Fi network. The local manager 104 may include input devices 218, such as buttons, a keyboard, a touchscreen, a microphone, etc. The local manager 104 also may include a modem for communicating over a telephone network, such as a cellular phone network.

The local manager 104 may be a dedicated custom device designed specifically for managing the medicament delivery device. One example of a such a dedicated device is a personal diabetes manager (PDM) for managing an insulin pump or other medicament delivery device for delivering insulin. Alternatively, as mentioned above, the local manager 104 may be realized on a smart phone or a mobile computing device, like a tablet or a wearable computing device.

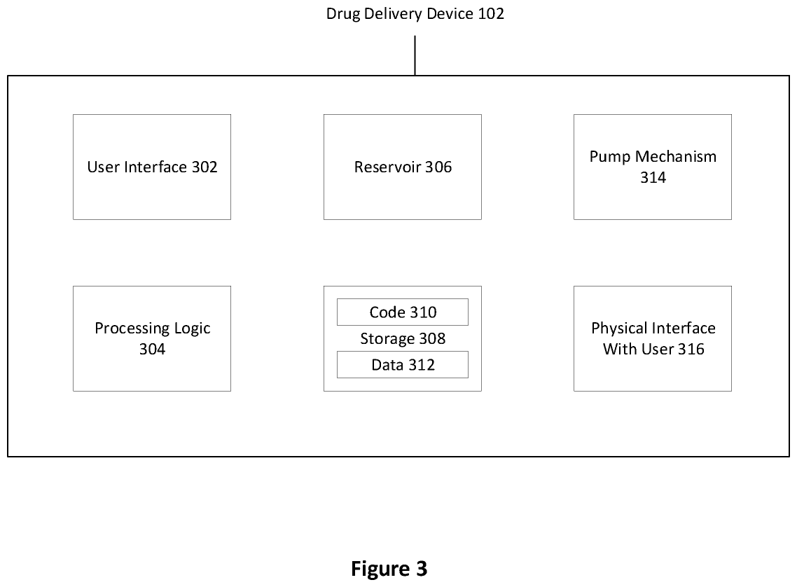

FIG. 3 depicts a block diagram of the medicament delivery device 102 showing more detail. The medicament delivery device 102 may include a user interface 302, such as a display, like a touch screen, and/or keys, buttons or other input mechanisms. The medicament delivery device 102 may include processing logic 304, such as a processor as described above, and/or electronic circuitry. The medicament delivery device 102 may include a reservoir 306 for storing the medicament to be delivered to the user 103. The medicament delivery device 102 may include a storage 308. The storage 308 may take any of the various forms of storage described above relative to the storage 208 of the local manger 104. The storage 308 may hold code 310 that is executable by the processing logic 304. The code 310 may include code for realizing a control method for the medicament delivery device 102. Where the medicament delivery device 102 delivers insulin, the code 310 may implement a control method for controlling the automatic delivery of insulin to the user 103. The storage 308 may also hold data 312 that may be used by the code 310. The medicament delivery device 102 may include a pump mechanism 314 for pumping the medicament out of the reservoir 306 on to the user 103 under the control of the control method. The medicament delivery device 102 additionally may include a physical interface with the user 316. The physical interface 316 may include needles, a cannula, tubes and the like for enabling the medicament to be delivered to the user 103.

As was discussed above, the remote manager 110 enables remote management of the medicament delivery device 102. One type of remote management is management of settings for the medicament delivery device 102. The remote manager can manage an adaptivity rate for the medicament delivery device 102 of the user 103. In this context, the adaptivity rate determines how quickly the control method seeks to achieve a desired outcome. For example, where the medicament delivery device 102 is an insulin pump that delivers insulin, the desired outcome is a target glucose level. The control method in this illustrative case may be provided with the latest blood glucose level reading from the sensor 106. The sensor 106 may be a continuous glucose monitor (CGM) in such a case. The control method seeks to adjust the user's glucose level from the current level to the target level. The control method determines how much basal insulin should be delivered and when to achieve this target level. Traditionally, or in ideal circumstances, half of the total daily insulin for a patient is delivered as basal insulin and half is delivered by boluses delivered by a patient. In some instances, the control method controls solely the basal insulin delivery and in others, the control method controls both the basal insulin delivery and at least some, or all, of the bolus deliveries. For this illustrative case, the adaptivity rate refers to how quickly the control method seeks to adjust the glucose rate of the user. Delivering higher doses of insulin may achieve more quickly reaching the target glucose level but also poses a greater risk of overshoot and hypoglycemia. The control method may employ a cost function that balances these competing interests and adjust the cost function to achieve a higher or a lower rate of adaptivity.

One or more parameters can characterize behavior of the control method. These parameters can quantify the aggressiveness of the control method as reflected in the amount of insulin automatically delivered by the medicament delivery device per cycle, where a cycle may be a fixed increment of time at which the medicament delivery device may automatically deliver insulin to the patient. These parameters may be adapted over time as expressed by the following equation:

P(i)=(1−R)*P(i−1)+R*P′(i)

where P(i) is a parameter value at time i, P(i−1) is the value of the parameter from the previous time i−1, P′(i) is the newly proposed value of the parameter (i.e. a target) and R is a parameter that affects the rate of adaptivity. In other words, the previous value of the parameter P is updated based on the weighted average of the current value of the parameter P and the newly proposed value of the parameter P′(i).

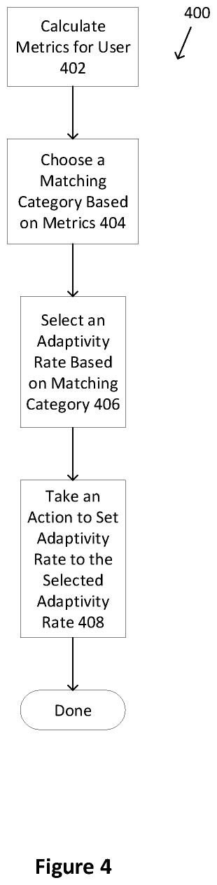

FIG. 4 depicts a flowchart 400 of illustrative steps that may be performed to adjust the adaptivity rate for the medicament delivery device 102 of a user based on data analytics by the remote manager 110. Metrics for the user are calculated from the data (402). These metrics may be calculated by the remote manager. The remote manager 110 has access to corresponding metrics for other users of the medicament delivery device. For instance, the metrics may specify information regarding the adaptivity of the users, including adaptivity rates. Based on the calculated metrics for the user, data analytics may be performed that compare the metrics and/or data for the user with similar metrics and/or data of the other users. Demographic, clinical, historical, and/or other information may be used to identify the similar other users. The data and metrics for those other users is examined and categorized into categories. One or more adaptivity rates may be associated with each category. A matching category may be chosen for the user based on the adaptivity characteristics (404). One of the adaptivity rates for the matching category may be chosen for the user (406). The remote manager 110 then may take an action to set the adaptivity rate for the medicament delivery device 102 of the user to the selected adaptivity rate (408).

In one exemplary embodiment, the metrics calculated in (402) are adaptivity characteristics. For a medicament delivery device that delivers insulin automatically in accordance with an automated insulin delivery control method, a first example of an adaptivity characteristic is a metric that measures the discrepancy between the amount of insulin delivered automatically and the total daily insulin for the user over a time period. This discrepancy may be expressed as:

$$D = \frac{{\sum }_{i = 1}^{{t}_{e n d}} U ( i )}{{TDI}_{I}}$$

where D is the discrepancy, i is an index of time for automated insulin deliveries over a day, U(i) is the amount of insulin automatically delivered at the time I, and TDI_{I} is the total daily insulin value when the medicament delivery device is activated. TDI includes the sum of basal insulin delivered and the sum of the amounts of insulin boluses that are delivered in the day, including, potentially, insulin delivered through an injection pen or other means.

A second illustrative adaptivity characteristic is the ratio of requested insulin boluses versus the insulin deliveries requested by the control method. This ratio may be expressed as:

$${Q}_{b o l u s} = \frac{{\sum }_{i = 1}^{{t}_{e n d}} {U}_{bolus} ( i )}{{\sum }_{i = 1}^{{t}_{e n d}} {U}_{a lgorith m} ( i )}$$

where Q_{bolus} is the ratio of requested insulin boluses versus insulin deliveries requested by the control method, i is an index of time for automated insulin deliveries over a day, U_{bolus}(i) is the amount of insulin bolused at time i and U_{algorithm}(i) is the amount of insulin delivered automatically by the control method at time i.

A third illustrative adaptivity characteristic is the ratio of time when the glucose level of the user fell below a low glucose threshold and the time that the control method suspended automatic delivery of insulin. This ratio can be expressed as:

$${Q}_{low , suspend} = \frac{{\sum }_{i = 1}^{{k}_{e n d}} {t}_{l o w} ( k )}{{\sum }_{i = 1}^{{k}_{e n d}} {t}_{susp} ( k )}$$

where Q_{low,suspend} is the ratio of the times, k is an index of time increments, t_{low}(k) is the time during the time increment k that the glucose level of the user was below the low threshold and tₛᵤₛₚ(k) is the time during the time increment k.

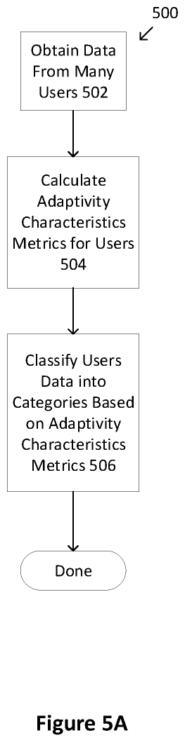

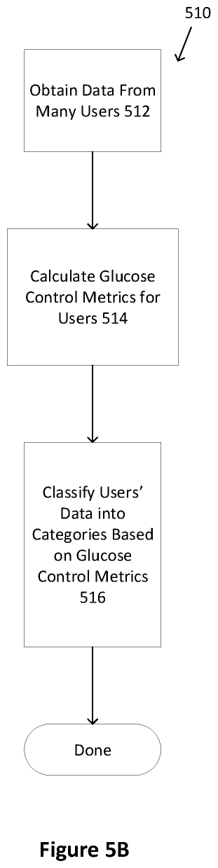

These three adaptivity characteristics may be used separately or in combination in the data analytics. Each represents a metric of mistuning of the settings of the control method versus the true needs of the user. Such adaptivity characteristics may be used in the method of the flowchart 400 of FIG. 4. Specifically, in (402) one of these metrics or a combination thereof may be calculated for the user. In (404) these adaptivity characteristics may be those that are used to choose a matching category. FIG. 5A depicts a flowchart 500 of what steps are performed to create the categories. First, data is obtained from multiple users of the medicament delivery device (502). Preferably the amount of data for the users and the number of users is large. From this data, the adaptivity characteristics metrics are calculated for the users (504). The resulting adaptivity characteristics for the users are classified into categories based on the metrics (506). The categories may be created by binning like values across users based on the adaptivity characteristics metrics. For example, the adaptivity characteristics can be binned based on the user's clinical parameters (e.g., TDI, basal insulin amount), clinical glucose control performance, physical locations (time zones), or demographics (e.g., age/gender). First, users who have high insulin requirements, as indicated by their clinical parameters, such as TDI or basal values, may be binned separately. In one example, this threshold may be above 60 U. Next, users who experience high glucose control performance metrics, such as a high % time in the 70-180 mg/dL range, may also be binned separately. In one example, this threshold may be above 80%. Further, users who are in different physical locations may share common characteristics and may be binned separately. In one example, this separation may be based on different time zones, or different patterns of changes in time zones, which may indicate users who experience extended periods of travel. Finally, users with different demographics may also be considered in separate bins. For instance, the adaptivity requirements of young children (<6 years old) may have significantly different characteristics than the adaptivity requirements of adults (18+ years old).

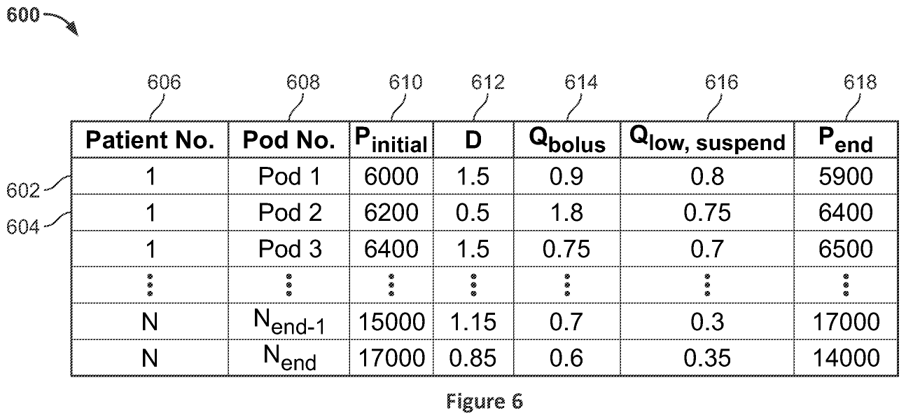

FIG. 6 depicts a table 600 holding values of the adaptivity characteristics and other data for users. Each row holds the data for a given instance of a device, such as a disposable medicament delivery pod, and user. Thus, row 602 holds data for Patient number 1 and pod number 1, whereas row 604 holds data for Patient number 1 and pod number 2. The columns are reserved for different types of data. Column 606 holds data identifying the patient number. Column 608 holds pod number data, and column 610 holds an initial P parameter value. Column 612 holds the discrepancy value D. Column 614 holds the ratio Q_{bolus}, and column 616 hold the ratio Q_{low, suspend}. Column 618 holds the final P value, P_{end}. Using this data for a category, the adaptivity rate as reflected in the parameter R can be set as:

R_{new}=R*min(2, (P_{end,best fit}/Pᵢₙᵢₜᵢₐₗ)

where R_{new} is the new value for R, min( ) is a function that selects a minimum, P_{end,best fit} is the value of P_{end} for the matching category and Pᵢₙᵢₜᵢₐₗ is the initial value of P.

The setting of the adaptivity rate need not be based on the adaptivity characteristics or the adaptivity characteristics alone. The setting of the adaptivity rate, for example, may be based on glucose outcomes where the medicament delivery device delivers insulin. In such an instance, the adaptivity rates may be categorized by glucose control outcomes. Since users may experience widely varying insulin needs, for some users there may be long delays before the control method results in stable values for the range of adaptivity parameters. If a single rate of adaptivity is applied for users of widely varying insulins needs, a slow rate of adaptivity conventionally may be chosen for all users. This results in less than ideal adaptivity rates for users who maintain good glucose level control. The exemplary embodiments may apply a sliding scale of adaptivity rates for users to produce more customized performance. The users that maintain good glucose have their adaptivity rate accelerated.

FIG. 5B depicts a flowchart 510 of illustrative steps that may be performed when the adaptivity rate is based on glucose level control outcomes. As before, data is obtained from many users (512). The glucose control metrics are calculated for the many users (514). The metrics used to categorize may be for example percentage of time in a desired range (such as a 70-180 mg/dL glucose concentration range), mean glucose value, etc. The users' data is then classified into categories based on the glucose control metrics (516). In such a case, the glucose control metrics for the user are calculated in (402) and the matching category of (404) is determined based on the categories determined in (516).

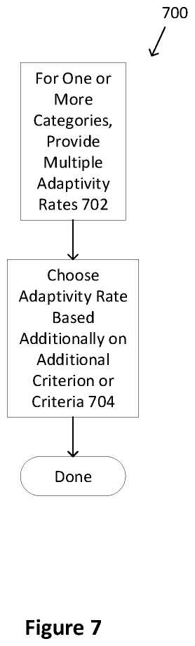

The selection of the adaptivity rate need not be based on a single metric or criteria for a user. In addition, a category need not have a single adaptivity rate associated with the category. FIG. 7 depicts a flowchart 700 of illustrative steps that may be performed in such instances. Multiple adaptivity rates are provided for more than one category (702). As was discussed above, categories may be defined based on metrics of adaptivity characteristics, glucose control outcomes, etc. One or more of such categories may have multiple associated adaptivity rates. The adaptivity rate for the user is chosen from among those associated with a category based on an additional criterion or criteria (704).

Examples of such additional criteria include date, time or a combination thereof. For example, a different adaptivity rate may be used for holidays than is used for days that are not holidays. Adaptivity rates may be chosen based on whether it is a weekend day or a weekday. Adaptivity rates may be chosen based on time of day or day of the week. Still further adaptivity rates may be chosen based on season or month.

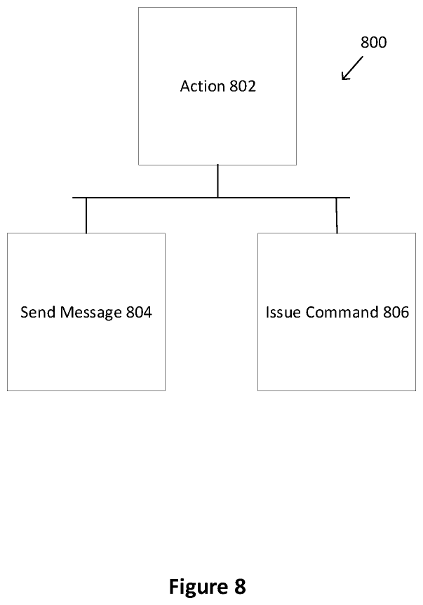

The action taken in (408) may take different forms. As shown in the diagram 800 of FIG. 8, an action 802 may take the form of sending a message 804. For example, the remote manager 110 may send a message to the local manager 104. The local manager 104 may respond to the message with a communication to the medicament delivery device 102 or may simply display the message on display 214 or display other content responsive to the received message. The message may direct the local manger 104 to take an action. The action 802 instead may be a command 806. For instance, the command may be to establish initial adaptivity rate settings or to adjust existing adaptivity rate settings.

The remote manager 110 is not limited in its managerial role to establishing or adjusting adaptivity rate settings. The remote manger 110 may perform a wide variety of management functions. Some of these functions rely upon information being provided regarding a user 103 and/or the medicament delivery device 102 of the user 103. In some instances, information may be intentionally solicited from the user 103 to tailor settings and activity of the medicament delivery device 102 for the user 103 as will be described below.

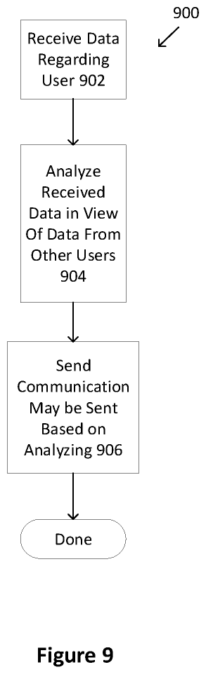

FIG. 9 depicts a flowchart of illustrative steps that may be performed in some exemplary embodiments by the remote manager 110. First, the remote manager receives data regarding the user (902). The data may be solicited or unsolicited and may be sent to the remote manager 110 from the local manager 104. The received data may be analyzed in view of data from other users (904). For example, data analytics may be performed on the data, like, for instance, the data regarding the user may be compared to that of the other users. Pattern matching may be performed or thresholding may be performed. Empirical norms, averages and other statistical values may be calculated and used. Based on the analysis, a communication is sent (906) from the remote manager 110 to the local manager 104. The communication may prompt action, may contain one or more commands or may be advisory or informative.

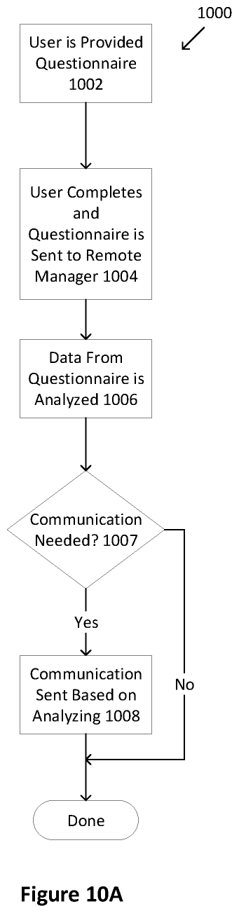

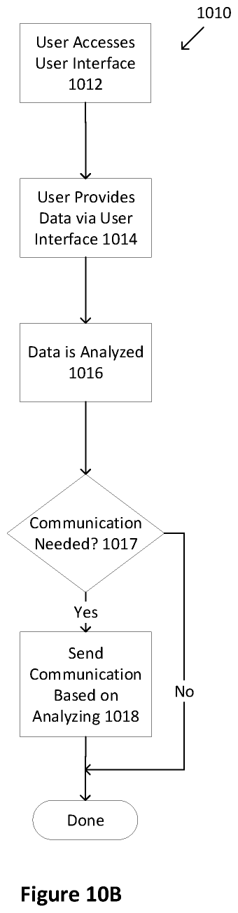

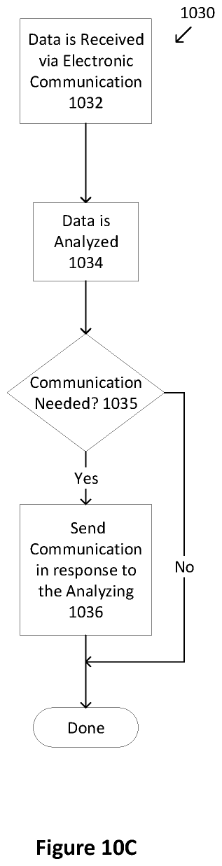

As was mentioned above, the data received in (902) may be prompted. One useful way to obtain data from a user is as shown in the flowchart 1000 of FIG. 10A to provide a questionnaire for the user to complete (1002). This may entail, as shown in the flowchart 1000 of FIG. 10, providing the user with an electronic questionnaire form to complete or may entail directing the user to a website that has a questionnaire to complete. Alternatively, an application may run on the local manager 104 that the user may use to complete the questionnaire. The user completes the questionnaire, and the questionnaire is sent to the remote manager 110 (1004). Data from the questionnaire is analyzed at the remote manager 110 or on the remote manager's behalf (1006). Based on the analysis, a determination is made whether a communication is needed or not (1007). If the determination is that a communication is needed, a communication is sent (1008) and otherwise is not.

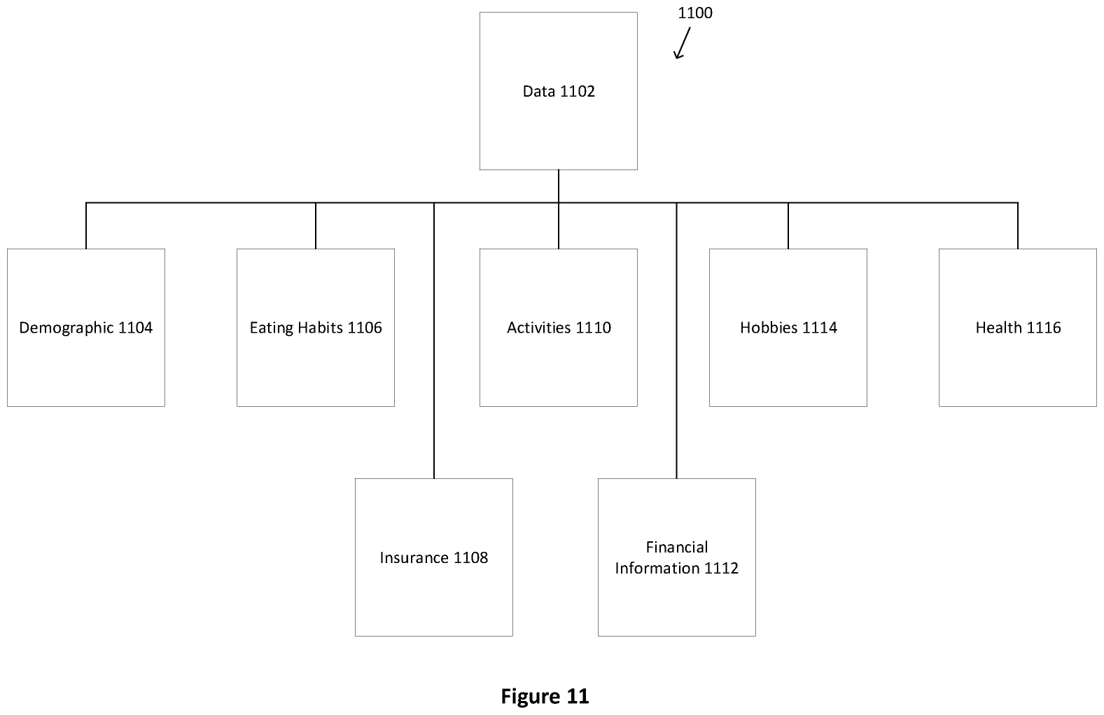

FIG. 11 depicts a diagram 1100 of potential types of data 1102 that may be of interest to the remote manager 110 and which may be provided, for example, by way of questionnaire. This data 1102 may be useful in creating a profile of the user and may be useful in deciding what other users are similar to the user. The data 1102 may include demographics data 1104, such as age, sex, residence, weight, height, etc. The data 1102 may including data regarding eating habits 1106, such as when the user eats, how much the user eats and how many carbohydrates are consumed. The data 1102 may include data about what kind of medical insurance coverage 1108 the user has. The type of insurance coverage that a user has may affect how often the user visits a medical professional and whether the user intentionally under-delivers the medicament to save money, etc. The data 1102 may include data regarding activities of the user 1110. For instance, it may be useful to know if the user exercises, how frequently the user exercises and at what intensity. The data 1102 may also include financial information 1112, such as average annual income. The data 1102 may include data regarding hobbies 1114. The data 1102 may include data regarding the health of the user 1116. For example, the data may indicate if the user has any health problems, any history of health problems, any allergies, etc.

Data 1102 may also be obtained via a user interface. For example, a user interface may be provided on a website for the remote manager 110. Alternatively, the local manager 104 may have a user interface for the user to enter data. In such a case, the user accesses the user interface (1012) (see the flowchart 1010 of FIG. 10B). The user enters data via the user interface (1014). The data is then analyzed at the remote manager 110 or on behalf of the remote manager (1016). A determination is made whether a communication needs to be sent or not (1017). If so, a communication is sent (1018). If not, no communication is sent.

The data also may be sent to the remote manager 110 by way of an electronic communication (e.g., message, etc.). FIG. 10C shows a flowchart 1030 of steps that may be performed in such an instance. Initially, data is received via an electronic communication (1032). For example, the local manager 104 may send an electronic communication to the remote manager 110. The electronic communication may either be in the message or may contain a reference to the data that may be used to gain access to the data. The data is then analyzed (1034), such as has been described above. Based on the analysis, a determination is made whether the remote manager 110 needs to send out a communication (1035). Based on the determination, a communication may be sent out (1036) or not.

The data provided may be used to build a profile of the user. This profile may be used to guide management of the medicament delivery device for the user. For instance, the profile may be used to establish initial settings and modify settings for the medicament delivery device 102 as needed. The data may also be used to prompt action, such as sending a command to bolus an amount of insulin because the received data indicates that the user is about to be hyperglycemic. Alternatively, the data may indicate that the user is at risk of hypoglycemia, and therefore, the remote manager 110 sends a communication to suspend delivery of insulin by the medicament delivery device 102.

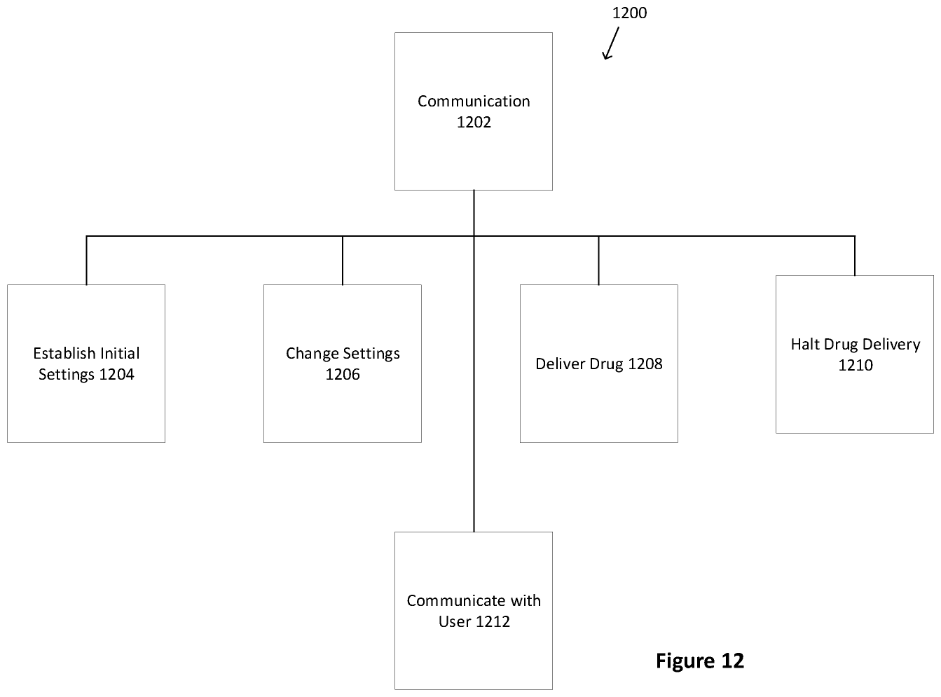

As shown in the diagram 1200 of FIG. 12, the communication sent from the remote manager 110 to the local manager 104 may prompt different effects. For instance, the communication 1202 may result in establishment of initial settings 1204 for the medicament delivery device 102. Alternatively, the communication 1202 may cause a change in settings 1206 for the medicament delivery device 102. As another alternative, the communication 1202 may simply serve as a communication with the user 1212 to provide information, such as advice, direction or an alert. The communication 1202 may result in delivery of a medicament 1208 to the user 103 via the medicament delivery device. Further, the communication may result in the halting of medicament delivery of the medicament 1210 to the user 103 from the medicament delivery device 102.

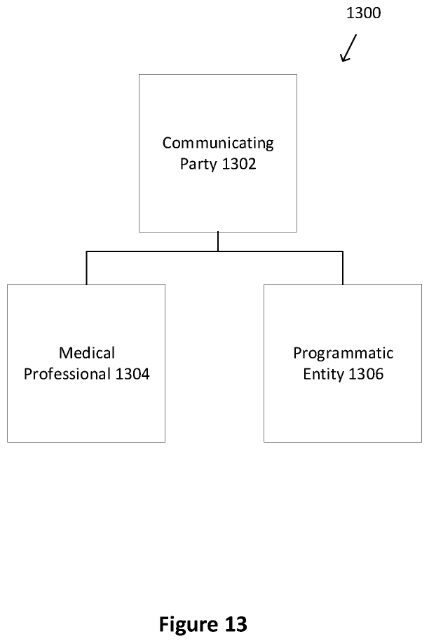

As shown in diagram 1300 of FIG. 13, a medical professional 1304, like a doctor, nurse, physician's assistant, pharmacist, etc., may be the source of the communications. The medical professional may review the data received, the user's profile or the results of the data analytics and generate a communication as has been described above. The remote manager 110 may have a user interface that enables the medical professional to create and send such a communication. Alternatively, the communicating party 1302 that sends the communication from the remote manager 110 may be a programmatic entity 1306, such as a computer program, module, object, routine, library, application, applet or other programmatic entity. The programmatic entity 1306 may have the intelligence for performing the data analytics, making a decision whether a communication is warranted and generating and sending the appropriate communication.

While the present invention has been described with reference to exemplary embodiments herein, various change in form and detail may be made without departing from the scope as defined in the appended claims.

1. A method, comprising:
with a processor, identifying other users of a medicament delivery device for delivering a medicament that have adaptivity characteristics like a selected user of the medicament delivery device;
with the processor, selecting an adaptivity rate for one or more parameters of the medicament delivery device for the selected user based on data related to adaptivity rate for the identified users, wherein the one or more parameters play a role in regulating automatic medicament delivery by the medicament delivery device; and
with the processor, taking an action to set the adaptivity rate for the medicament delivery device to the selected adaptivity rate.

2. The method of claim 1, wherein taking an action comprises one of sending a communication to the medicament delivery device of the selected user or sending a command to set the adaptivity rate as the selected adaptivity rate for the medicament delivery device of the selected user.

3. The method of claim 1, wherein the selected adaptivity rate is for a single parameter.

4. The method of claim 1, wherein the medicament delivered by the medicament delivery device is insulin.

5. The method of claim 4, wherein the adaptivity characteristics are one or more of the following metrics: discrepancy between amount of insulin delivered automatically by the medicament delivery device for a day versus total daily insulin, a ratio of amount of insulin requested as boluses for a period versus an amount of automatically delivered insulin for the period or a ratio of a percentage of time where glucose is below a hypoglycemic threshold versus a percentage of time where automatic delivery of insulin was suspended.

6. The method of claim 1, further comprising:
categorizing the data related to adaptivity rate for the identified users into categories;
choosing a matching category among the categories for the selected user; and
using the data related to adaptivity rate for the identified users in the matching category in the selecting of the adaptivity rate for one or more parameters of the medicament delivery device for the selected user.

7. The method of claim 6, further comprising projecting an expected final value for the one or more parameters and determining an adaptivity rate based on how great of a difference there is between the expected final value and an initial value of the one or more parameters and wherein the determined adaptivity rate is the selected adaptivity rate.

8. The method of claim 6, wherein the categorizing categorizes the data by glucose control outcomes and wherein choosing a matching category comprises choosing the matching category as one that has a glucose control outcome that suits the selected user.

9. The method of claim 8, wherein the categorizing categorizes based on data including at least one of mean glucose value or percentage of time glucose value is in an acceptable range.

10. The method of claim 1, wherein the selecting an adaptivity rate for one or more parameters of the medicament delivery device for the selected user is also based on at least one of date, time, day of week or whether a day is a holiday or part of a holiday season.

11. The method of claim 1, further comprising updating the data related to adaptivity rate for the identified users before the selecting.

12. A method, comprising:
receiving information regarding a user of a medicament delivery device and/or the medicament delivery device at a remote computing device, wherein the remote computing device is not connected to a network that the medicament delivery device is connected; and
sending a communication to the medicament delivery device from the remote computing device causing at least one of the following: a medicament to be delivered by the medicament delivery device to the user, settings of the medicament delivery device to be changed, settings of a controller of the medicament delivery device to be changed or information to be sent to the user of the medicament delivery device.

13. The method of claim 12, wherein the communication is sent to the controller for the medicament delivery device.

14. The method of claim 12, wherein the medicament delivery device is an insulin delivery device that delivers insulin to the user.

15. The method of claim 12, further comprising displaying a user interface on the remote computing device for initiating the sending of the communication.

16. The method of claim 12, wherein the received information includes glucose information for the user.

17. The method of claim 12, further comprising displaying at least some of the received information on a display of the remote computing device.

18. The method of claim 12, wherein the method further comprises sending a questionnaire or providing a user interface to the user to solicit the information that is received.

19. The method of claim 18, wherein the solicited information includes at least one of information regarding activities of the user, information regarding hobbies of the user, information regarding eating habits of the user, information about health of the user, information on medicament delivery by the user, information regarding glucose history or demographic information about the user.

20. A non-transitory computer-readable storage medium storing instructions that when executed by a processor cause the processor to perform the following:
identify other users of a medicament delivery device for delivering a medicament that have adaptivity characteristics like a selected user of the medicament delivery device;
select an adaptivity rate for one or more parameters of the medicament delivery device for the selected user based on data related to adaptivity rate for the identified users, wherein the one or more parameters play a role in regulating automatic medicament delivery by the medicament delivery device; and
take an action to set the adaptivity rate for the medicament delivery device to the selected adaptivity rate.
